(19) United States
(12) Patent Application Publication
(10) Pub. No.: US 2011/0270284 A1
(43) Pub. Date: Nov. 3, 2011

(54) MUSCULO-SKELETAL MESH AND FIXATION SYSTEM

(75) Inventors: Christopher P. Beauchamp, Scottsdale, AZ (US); James M. Swain, Rochester, MN (US)

(73) Assignee: MAYO FOUNDATION FOR MEDICAL EDUCATION AND RESEARCH, Rochester, MN (US)

(21) Appl. No.: 12/572,770

(22) Filed: Oct. 2, 2009

Related U.S. Application Data
(60) Provisional application No. 61/102,544, filed on Oct. 3, 2008.

(51) Int. Cl. A61B 17/08

(52) U.S. Cl. 606/151

(57) ABSTRACT

A surgical mesh product and a method for attaching the surgical method product to a patient's bodily tissue. The surgical mesh product includes a fixation structure can be a grommet, an interwoven fiber or thread, or a reinforcing material. The fixation structure strengthens the mesh material (e.g. enhances the rip and/or tear strength) at locations where screws, tacks or other fasteners will be applied to secure the mesh product to a patient. The surgical mesh product is suitable for use in hernia repair.

### CROSS-REFERENCE TO RELATED APPLICATION

The present application claims the benefit of U.S. Provisional Application 61/102,544, filed Oct. 3, 2008, which is incorporated herein by reference in its entirety for all purposes.

### TECHNICAL FIELD

The invention is a surgical mesh product of the type that can be used for hernia repair, and a method for attaching the mesh to tissue.

### BACKGROUND OF THE INVENTION

Surgical mesh products are commonly used to repair hernia, including those located immediately adjacent to bone structures. Examples of anatomic locations include hernia occurring after prostatectomy, hysterectomy and other pelvic surgery and after iliac crest harvesting for bone grafting. Mesh of this type and related surgical procedures are disclosed, for example, in the following patents and published applications.

| Inventor Name | Patent/Publication No. |
|---|---|
| Kieturakis et al. | U.S. Pat. No. 5,464,403 |
| Titone et al. | U.S. Pat. No. 5,569,273 |
| Kugel | U.S. Pat. No. 5,634,931 |
| Tormala et al. | U.S. Pat. No. 6,319,264 |
| Zotti et al. | U.S. Pat. No. 7,094,261 |
| Eisermann et al. | US 2002/0123750 |
| Criscuolo et al. | EP 1 721 575 |
| Williams | WO 2006/119216 |

Unfortunately, known surgical meshes can be difficult to fix to some locations. Attachment and healing to bone is, for example often poor. There is, therefore, a continuing need for improved surgical mesh products and associated methods of fixing the mesh to bone and other tissue.

### SUMMARY OF THE INVENTION

According to various embodiments, the invention is a surgical mesh product including a mesh material and one or more fixation structures. The fixation structures can include grommets, fibers or threads that are interwoven or otherwise embedded in the mesh material, or a strip of reinforcing material that is overlayed and attached to the mesh material.

According to other embodiments, the invention is a multi-layered surgical mesh product. The surgical mesh product includes a first material layer, a second material layer and one or more fixation structures.

According to yet another embodiment, the invention is a procedure of using a mesh product including a mesh material and one or more fixation structures including the step of attaching portions of the mesh product having the fixation structures to bone.

### BRIEF DESCRIPTION OF THE DRAWINGS

FIG. 1 is a schematic illustration of a mesh product provided in accordance with one embodiment of the invention.

FIG. 2 is a schematic illustration of a mesh product provided in accordance with a second embodiment of the invention.

FIG. 3 is a schematic illustration of a mesh product provided in accordance with yet another embodiment of the invention.

FIG. 4 is a side view of a multi-layer mesh product provided in accordance with still another embodiment of the invention.

FIGS. 5A and 5B are schematic illustrations of a portion of a patient's body with mesh product deployed to repair a hernia at a junction of bone and muscle where the muscle has pulled away from the bone in accordance with still another embodiment of the invention.

### DETAILED DESCRIPTION

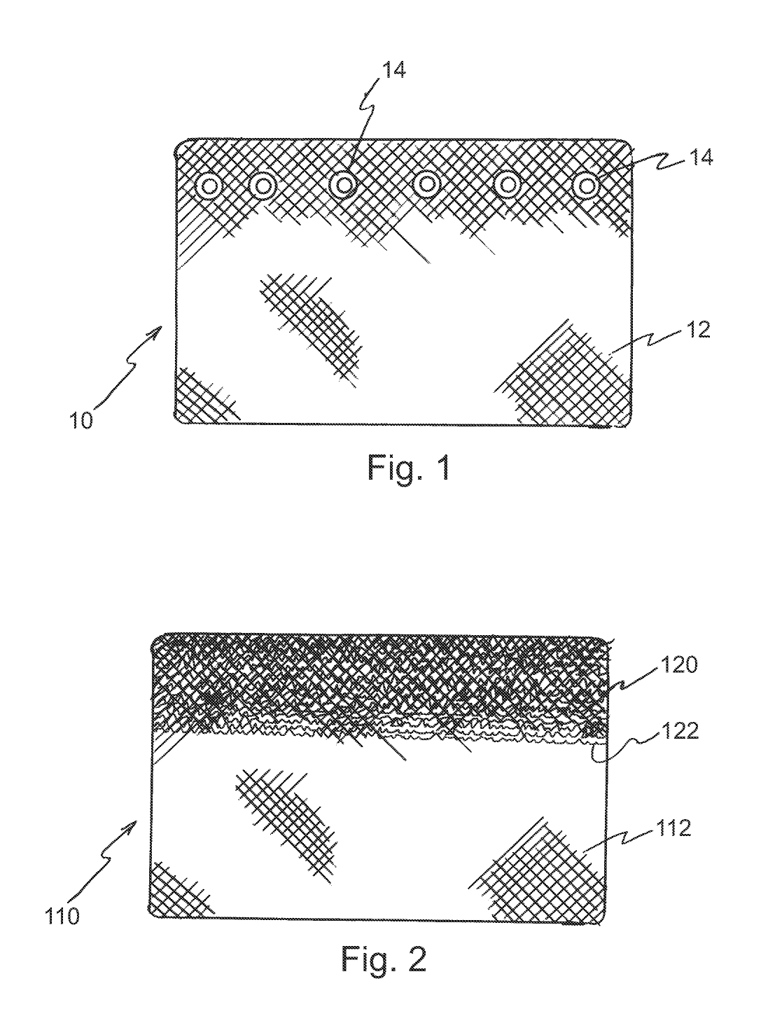

FIG. 1 is an illustration of a mesh product 10 in accordance with one embodiment of the invention. As shown, mesh product 10 includes a piece of mesh material 12 and one or more grommets 14 (six are shown) attached to the mesh material as fixation structures. The mesh material 12 can be any material suitable for the intended application, including PTFE (polytertafluoroethylene), prolene (polypropolene), Vypro (polyglactin and polypropylene) and bio-derived materials (such as porcine submucosa, biopolymers). Grommets 14 can be of any conventional design and applied to the mesh material 12 either before or during the surgical procedure. The grommets 14 can be formed of biocompatible polymers such as those mentioned above, or metals such as stainless steel and nitinol. In the embodiment shown, grommets 14 are positioned at spaced-apart locations along one edge of the mesh material 12. In other embodiments (not shown) the grommets 14 are positioned at other or additional locations. As described below, screws, tacks or other surgical fasteners are inserted through the grommets 14 to secure the mesh product 10 to bone or other tissue of the patient.

FIG. 2 is an illustration of a mesh product 110 in accordance with a second embodiment of the invention. As shown, mesh product 110 includes a piece of mesh material 112 having a fixation structure 120. The fixation structure 120 includes fibers or threads 122 that are interwoven or otherwise embedded in the mesh material 112. Threads 122 can be polymers of the types used in the mesh material 112 or metals such as stainless steel and nitinol. In the illustrated embodiment the fixation structure 120 is positioned along one edge of the mesh material 112. In other embodiments (not shown) the fixation structure 120 is positioned at other and/or additional locations. The fixation structure 120 strengthens the mesh material 112 (e.g., enhances the rip and/or tear strength) at locations where screws, tacks or other fasteners will be applied to secure the mesh product 110 to a patient.

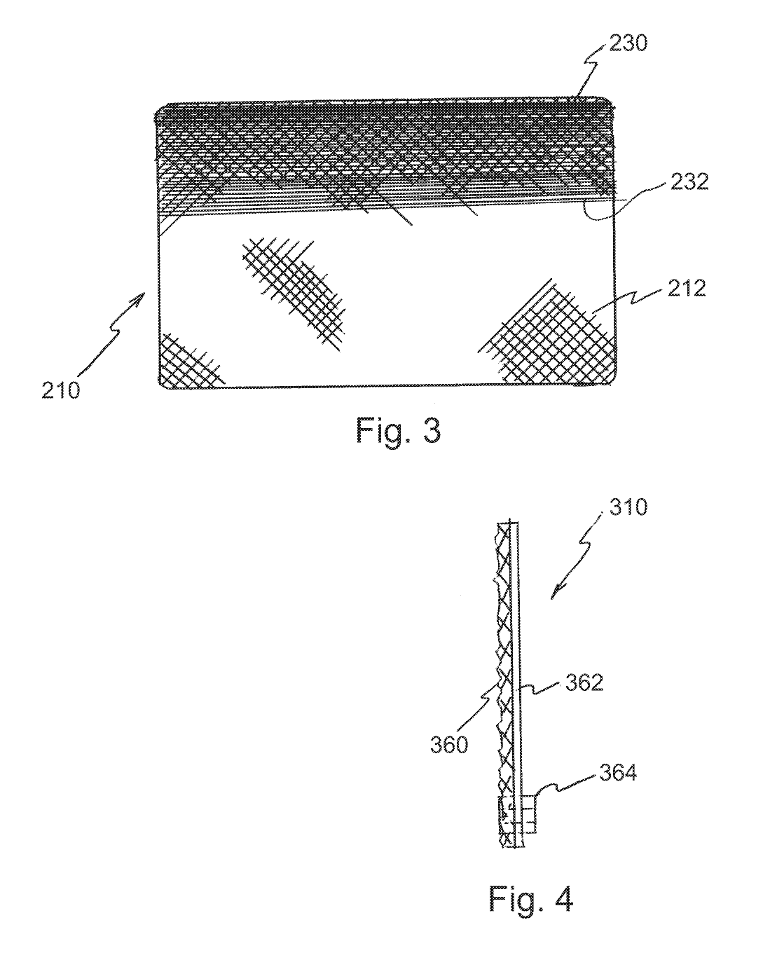

FIG. 3 is an illustration of a mesh product 210 in accordance with yet another embodiment of the invention. As shown, mesh product 210 includes a piece of mesh material 212 having a fixation structure 230. The fixation structure 230 includes a strip of reinforcing material 232 that is overlayed and attached to the mesh material 212 by adhesive or other structures such as stitching or mechanical fasteners. The reinforcing material 232 can be woven on nonwoven polymer materials, metal, combinations of polymer and metal or other suitable materials. In the illustrated embodiment the fixation structure 230 is positioned along one edge of the mesh material 212. In other embodiments (not shown) the fixation structure 230 is positioned at other and/or additional locations. The fixation structure 230 strengthens the mesh material 212 (e.g., enhances the rip and/or tear strength) at locations where screws, tacks or other fasteners will be applied to secure the mesh product 210 to a patient.

FIG. 4 is a side view of a multi-layer mesh product 310 in accordance with still another embodiment of the invention. Mesh product 310 has a first material layer 360, a second material layer 362 and one or more fixation structures such as grommet 364 (only one is shown). First material layer 360 has a fiberous, porous or roughened external surface to promote tissue ingrowth when deployed and fixed to tissue such as bone and muscle. First material layer 360 can be mesh material or other materials of the embodiments of the invention described above in connection with FIGS. 1-3. Other materials can also be used. The second material layer 362 is joined to the first material layer (e.g., by laminating, adhesives or other processes or structures. The external surface of the second material layer 362 is relatively smooth and low-friction to resist or prevent tissue ingrowth and adhesion. PTFE or other materials can be used for the second material layer 362. In other embodiments, drugs or other substances that resist tissue ingrowth can be applied to the material. In still other embodiments (not shown), mesh product 310 is formed from a single layer of material, but its opposite surfaces have the characteristics of the first and second materials 360 and 262, respectively, described above.

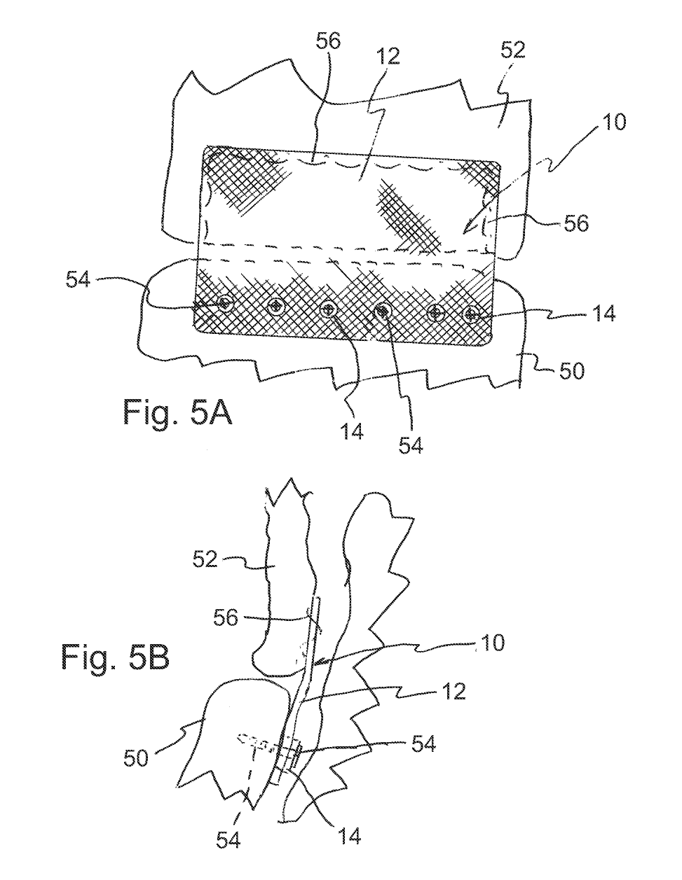

FIGS. 5A and 5B are illustrations of a portion of a patient's body with mesh product 10 deployed to repair a hernia at a junction of bone 50 and muscle 52 where the muscle has pulled away from the bone. As shown, the portion of the mesh product 10 having the grommets 14 is positioned against the bone 50 and screws 54 are inserted into the grommets and screwed into the bone. Mesh material 12 is thereby securely fastened to the bone 50. Portions of the mesh product 10 adjacent to the muscle 52 (and free from fixation structures in the illustrated embodiment) are attached to the muscle by sutures 56 or other approaches (e.g., staples, not shown). The grommets 14 can be fixed to the material 12 before the mesh product 10 is implanted in the patient. Alternatively, tools (not shown) can be used to attach the grommets 14 to the mesh material 12 after the material is implanted in the patient but before it is attached to the tissue. The mesh product 10 can thereby be customized or tailored to the patient's anatomy.

Conventional or minimally invasive surgical procedures and associated instruments and tools (e.g., laparoscopic screw drivers) can be used to perform these surgical operations. The mesh products 110, 210 and 310 described above in connection with FIGS. 2-4 can be deployed using similar procedures and tools. In particular, the fixation structures such as 120, 230 and 364 of these mesh products can be attached to the bone of the patient by fasteners such as screws and tacks.

The invention offers a number of advantages. The mesh product can, for example, be securely fastened to bone and other tissues of the patient. Ripping and tearing of the mesh from the tissue can be reduced. Healing is thereby enhanced.

Although the invention has been described with reference to preferred embodiments, those skilled in the art will recognize that changes can be made in form and detail without departing from the spirit and scope of the invention.

1. A surgical mesh product having one or more fixation structures.

2. The surgical mesh product of claim 1, wherein the one or more fixation structures are positioned along an edge of the mesh product.

3. The surgical mesh product of claim 2, wherein the fixation structures include grommets.

4. The surgical mesh product of claim 2, wherein the fixation structures include interwoven fibers or threads.

5. The surgical mesh product of claim 2, wherein the fixation structure includes reinforcing material.

6. A surgical procedure using a mesh product including a mesh material and one or more fixation structures comprising the step of attaching portions of the product having the fixation structures to bone.

7. The procedure of claim 6, wherein the step of attaching the fixation structures includes screwing or tacking the fixation structures to bone.

8. The procedure of claim 6, further including the step of attaching portions of the product free from the fixation structures to muscle.

9. The procedure of claim 8, wherein attaching the portions of the product to muscle includes suturing or stapling the products to bone.

10. A surgical mesh product comprising: a mesh material comprising a first material layer and a second material layer and one or more fixation structures.

11. The surgical mesh product of claim 10, wherein the first material layer comprises a surface adapted to promote tissue ingrowth when deployed and fixated to tissue.

12. The surgical mesh product of claim 10, wherein the second material is joined to the first material layer and has a smooth external surface adapted to prevent tissue ingrowth.

13. The surgical mesh product of claim 10, wherein the one or more fixation structures are positioned along an edge of the mesh product.

14. The surgical mesh product of claim 13, wherein the fixation structures include grommets.
